(19) United States
(12) Patent Application Publication
(10) Pub. No.: US 2005/0004412 A1
(43) Pub. Date: Jan. 6, 2005

(54) Distillation of a Fischer-Tropsch derived hydrocarbon stream

(75) Inventors: Richard O. Moore JR., San Rafael, CA (US); Donald L. Kuehne, Hercules, CA (US); Richard E. Hoffer, Windsor (GB)

Correspondence Address: BURNS, DOANE, SWECKER & MATHIS, L.L.P., P.O. Box 1404, Alexandria, VA, 22313-1404, US

(73) Assignee: Chevron U.S.A. Inc,

(21) Appl. No.: 10/613,423

(22) Filed: Jul. 2, 2003

(51) Int. Cl. C07C 7/00

(52) U.S. Cl. 585/802

(57) ABSTRACT

Novel methods of treating a Fischer-Tropsch product by distillation are disclosed. Such methods are capable of removing soluble (and ultra-fine particulate) contamination, fouling agents, and/or plugging precursors from the Fischer-Tropsch derived hydrocarbon stream such that plugging of the catalyst beds of a subsequent hydroprocessing step is substantially avoided.

### REFERENCE TO RELATED APPLICATIONS

The present application hereby incorporates by reference in its entirety U.S. patent application Ser. No. ______, entitled “Acid Treatment of a Fischer-Tropsch Derived Hydrocarbon Stream,” by Lucy M. Bull, William Schinski, Donald L. Kuehne, Rudi Heydenrich, and Richard O. Moore, Jr.; U.S. patent application Ser. No. ______, entitled “Catalytic Filtering of a Fischer-Tropsch Derived Hydrocarbon Stream,” by Jerome F. Mayer, Andrew Rainis, and Richard O. Moore, Jr.; and U.S. patent application Ser. No. ______, entitled “Ion Exchange Methods of Treating a Fischer-Tropsch Derived Hydrocarbon Stream,” by Lucy M. Bull and Donald L. Kuehne.

### BACKGROUND OF THE INVENTION

1. Field of the Invention

The present invention relates in general to the hydroprocessing of products from a Fischer-Tropsch synthesis reaction. More specifically, embodiments of the present invention are directed toward a distillation procedure designed to effectively remove contamination from a Fischer-Tropsch derived hydrocarbon stream prior to passing that hydrocarbon stream on to additional processing.

2. State of the Art

The majority of the fuel used today is derived from crude oil, and crude oil is in limited supply. However, there is an alternative feedstock from which hydrocarbon fuels, lubricating oils, chemicals, and chemical feedstocks may be produced; this feedstock is natural gas. One method of utilizing natural gas to produce fuels and the like involves first converting the natural gas into an “intermediate” known as syngas (also known as synthesis gas), a mixture of carbon monoxide (CO) and hydrogen (H₂), and then converting that syngas into the desired liquid fuel(s) using a process known as a Fischer-Tropsch (FT) synthesis. A Fischer-Tropsch synthesis is an example of a so-called gas-to-liquids (GTL) process since natural gas is converted into a liquid fuel. Typically, Fischer-Tropsch syntheses are carried out in slurry bed or fluid bed reactors, and the hydrocarbon products have a broad spectrum of molecular weights ranging from methane (C₁) to wax (C₂₀₊).

The Fischer-Tropsch products in general, and the wax in particular, may then be converted to products including chemical intermediates and chemical feedstocks, naphtha, jet fuel, diesel fuel, and lubricant oil basestocks. For example, the hydroprocessing of Fischer-Tropsch products may be carried out in a trickle flow, fixed catalyst bed reactor wherein hydrogen (H₂), or a hydrogen enriched gas, and the Fischer-Tropsch derived hydrocarbon stream comprise the feed to the hydroprocessing reactor. The hydroprocessing step is then accomplished by passing the Fischer-Tropsch derived hydrocarbon stream through one or more catalyst beds within the hydroprocessing reactor with a stream of the hydrogen enriched gas.

In some cases, the feeds to be hydroprocessed contain contaminants that. originate from upstream processing. These contaminants may take either a soluble or particulate form, and include catalyst fines, catalyst support material and the like, and rust and scale from upstream processing equipment. Fischer-Tropsch wax and heavy products, especially from slurry and fluid bed processes, may contain particulate contaminants (such as catalyst fines) that are not adequately removed by filters provided for that purpose. The removal of those particulates prior to hydroprocessing may be complicated by the potentially high viscosities and temperatures of the wax stream leaving the Fischer-Tropsch reactor.

The typical catalyst used in a hydroprocessing reactor demonstrates a finite cycle time; that is to say, a limited time (or amount) of usefulness before it has to be replaced with a new catalyst charge. The duration of this cycle time usually ranges from about six months to four years or more. It will be apparent to one skilled in the art that the longer the cycle time of a hydroprocessing catalyst, the better the operating efficiency of the plant.

Soluble and/or particulate contaminants can create serious problems if they are introduced into the hydroprocessing reactor with the feed. The soluble contaminants pose a problem when, under certain conditions of hydroprocessing, they precipitate out of solution to become particulates. The contamination can cause partial or even complete plugging of the flow-paths through the catalyst beds as the contamination accumulates on the surfaces and interstices of the catalyst. In effect, the catalyst pellets filter out particulate contamination from the feed. In addition to trapping debris that is entrained in the feed, the catalyst beds may also trap reaction by-products from the hydroprocessing reaction itself, an example of such a reaction by-product being coke. Plugging can lead to an impairment of the flow of material through the catalyst bed(s), and a subsequent buildup in the hydraulic pressure-drop across the reactor (meaning the pressure differential between the ends of the reactor where the entry and exit ports are located, respectively). Such an increase in pressure-drop may threaten the mechanical integrity of the hydroprocessing reactor internals.

There are at least two potentially undesirable consequences of catalyst bed plugging. One is a decrease in reactor throughput. A more serious consequence is that a complete shut down of the reactor may be required to replace part or all of the catalyst charge. Either of these consequences can have a negative effect on operating plant economics.

Prior art attempts to manage the problem of catalyst bed plugging in hydroprocessing reactors have been directed toward eliminating at least a portion of the particulate contamination in the feed by filtering the feed prior to its introduction to the hydroprocessing reactor. Such conventional filtration methods are usually capable of removing particulates larger than about 1 micron in diameter. Other prior art methods have been directed toward either controlling the rate of coking on the hydroprocessing catalyst, selecting a feed that is not likely to produce coke, or judiciously choosing the hydroprocessing conditions (conditions such as hydrogen partial pressure, reactor temperature, and catalyst type) that affect coke formation.

The present inventors have found, however, that the above-mentioned open art methods are not effective at removing very small sized particle (or soluble) contaminants, fouling agents, and/or plugging-precursors (hereinafter referred to as “contamination”) from the feedstream to a hydroprocessing reactor when that feedstream comprises a Fischer-Tropsch derived hydrocarbon stream. This is particularly true when the Fischer-Tropsch derived hydrocarbon stream is a wax produced by a slurry bed or fluid bed process. Typical open art methods have therefore not been found to be effective at avoiding the pressure-drop buildup in a hydroprocessing, hydroisomerization, or hydrotreating reactor when that buildup is caused either by particulate contamination, or by soluble contamination that precipitates out of solution.

The apparent failure of typical open art methods has been attributed to either the presence in the hydroprocessing reactor feed of finely divided, solid particulates with diameters of less than about one micron, and/or to a soluble contaminant, possibly having a metallic component, with the ability to precipitate out of solution adjacent to or within the hydroprocessing reactor catalyst beds. What is needed is a method of removing particulates, contaminants, soluble contamination, fouling agents, and plugging precursors from the feedstream to a hydroprocessing reactor such that pressure drop buildup within the hydroprocessing reactor is substantially avoided.

### SUMMARY OF THE INVENTION

A Fischer-Tropsch synthesis is an example of a so-called gas-to-liquids (GTL) process, where natural gas is first converted into syngas (a mixture substantially comprising carbon monoxide and hydrogen), and the syngas is then converted into the desired liquid fuels. Typically, Fischer-Tropsch syntheses are carried out in slurry bed or fluid bed reactors, and the hydrocarbon products have a broad spectrum of molecular weights ranging from methane (C₁) to wax (C₂₀₊). The Fischer-Tropsch products in general, and the wax in particular, may then be hydroprocessed to form products in the distillate fuel and lubricating oil range. According to embodiments of the present invention, hydroprocessing may be conducted in either an upflow or downflow mode. The present process is particularly applicable to operation in the downflow mode.

In some cases, the feeds to be hydroprocessed contain contamination that originates from upstream processing. This contamination may include catalyst fines, catalyst support material and the like, and rust and scale from upstream processing equipment. Fischer-Tropsch wax and heavy products, especially from slurry and fluid bed processes, may contain contamination (such as catalyst fines) that is not adequately removed by filters provided for that purpose. Contamination can create a serious problem if it is introduced into the hydroprocessing reactor with the feed. The contamination can cause partial or even complete plugging of the flow-paths through the catalyst beds as the contamination accumulates on the surfaces and interstices of the catalyst.

The present inventors have found new methods that are effective at removing contamination, which may include particulates, solidified contaminants, soluble contamination, fouling agents, and/or plugging-precursors from the feed stream to a hydroprocessing reactor when that feed comprises a Fischer-Tropsch derived hydrocarbon stream. The consequences of contamination in the Fischer-Tropsch derived hydrocarbon stream typically include a pressure-drop buildup in the hydroprocessing reactor.

Embodiments of the present invention are directed to the removal of such contamination by distillation. The inventors have discovered that distillation of a Fischer-Tropsch derived hydrocarbon stream can substantially reduce plugging of a hydroprocessing reactor when the distillation is performed prior to the introduction of that hydrocarbon stream to the hydroprocessing reactor. The distillation may~be performed in either a batch or continuous mode. A single distillation operation can substantially decrease the plugging problem, but in one embodiment of the present invention, a double distillation process is advantageous. A double distillation process can remove substantially all of the contamination, and reduce or eliminate the plugging of the hydroprocessing reactor such that hydroprocessing catalyst cycle times are restored to their desired two to four year or more duration.

In one embodiment of the present invention, contamination is removed from a Fischer-Tropsch derived hydrocarbon stream using the steps:
a) filtering a Fischer-Tropsch derived hydrocarbon stream to produce a filtered hydrocarbon stream;
b) passing the filtered hydrocarbon stream to at least one distillation step to remove contamination from the filtered hydrocarbon stream, the distillation step producing a distillate product stream and a bottoms fraction, wherein the contamination is substantially concentrated in the bottoms fraction; and
c) recovering the bottoms fraction from the distillation step, wherein the amount of the bottoms fraction is less than about 35 percent by volume of the filtered hydrocarbon stream.

In other embodiments the amount of the bottoms fraction is less than about 15, 10, and 5 percent by volume of the filtered stream, respectively.

Other embodiments of the present invention are directed to methods of distilling a Fischer-Tropsch derived hydrocarbon stream to remove contamination, wherein the distillation step comprises a first distillation step and a second distillation step, the first distillation step producing a first overhead stream and a first bottoms stream, and wherein the second distillation step produces a second overhead stream and a second bottoms stream. The first overhead stream may be passed to the hydroprocessing reactor, the first bottoms stream may be passed to the second distillation step, and the second overhead stream may be passed to the hydroprocessing reactor.

The present embodiments may further include the steps of treating the second bottoms stream with a treatment selected from the group consisting of dispensing the second bottoms stream in crude oil, passing the second bottoms stream to a third distillation step, processing the second bottoms stream into a fuel, and recycling the second bottoms stream in a recycling operation.

The present methods include a distillation process that removes contamination from a Fischer-Tropsch derived hydrocarbon stream. The contamination may be in either particulate or soluble form, and may be derived from a Fischer-Tropsch catalyst comprising a material selected from the group consisting of aluminum, cobalt, titanium and iron. According to the present methods, the distillation process substantially avoids plugging of hydroprocessing catalyst beds in a hydroprocessing reactor such that the cycle time of the hydroprocessing catalyst is at least two years.

### BRIEF DESCRIPTION OF THE DRAWINGS

FIG. 1 is a block diagram showing an overview of embodiments of the present invention;

FIG. 2 is a schematic diagram showing a process flow according to embodiments of the present invention, including the filtering of at least a portion of the products of a Fischer-Tropsch reaction to produce a filtered product, a distillation of the filtered product, and the hydroprocessing of at least a portion of the distilled Fischer-Tropsch derived stream;

FIG. 3 is a schematic diagram of an embodiment of the present invention in which two successive distillation processes are carried out on the Fischer-Tropsch hydrocarbon stream;

FIG. 4 is an overview of an exemplary batch distillation process used to concentrate the contamination into a bottoms fraction; and

FIG. 5 is an overview of an exemplary continuous distillation process used to concentrate the contamination into a bottoms stream.

### DETAILED DESCRIPTION OF THE INVENTION

Embodiments of the present invention are directed to the hydroprocessing of products from a Fischer-Tropsch synthesis reaction. The present inventors have observed under certain conditions a tendency for the catalyst beds in a hydroprocessing reactor to become plugged by either particulate contamination, or by soluble contaminants that precipitate out of solution in the vicinity of or within the catalyst beds, thus impeding the flow of material through the hydroprocessing reactor. The contamination may still be present (meaning the problem still exists) even when the Fischer-Tropsch derived hydrocarbon stream is filtered to remove particulate debris larger than about 0.1 microns.

Though not wishing to be bound by any particular theory, the inventors believe the contamination may be present (at least partly) in the Fischer-Tropsch derived hydrocarbon stream in a soluble form, and the contamination may then precipitate out of solution to form solid particulates after the stream is charged to, for example, a hydroprocessing reactor. The contamination may or may not originate from a foreign source. Typically, after precipitating, the contamination forms solid plugs in the hydroprocessing reactor. Under certain conditions, the plugging occurs in a central portion of the reactor. The spatial extent of the plugging depends on hydroprocessing conditions and catalyst type, where varying space velocities, for example, can compress or spread the plugging over and/or into different regions of the reactor. Whatever its form, the contamination is an undesirable component(s) in the context of hydroprocessing, since it has the potential to plug the flowpaths through the hydroprocessing reactor.

While it is not certain whether the contamination is present in the Fischer-Tropsch derived hydrocarbon stream as a soluble species, or as an ultra-fine particulate (meaning probably less than about 0.1 microns in size), it is known that the contamination is not generally removed from that hydroprocessing feedstream by conventional filtering.

The inventors have discovered that distillation of a Fischer-Tropsch derived hydrocarbon stream can substantially reduce plugging of a hydroprocessing reactor when the distillation is performed prior to the introduction of the Fischer-Tropsch derived hydrocarbon stream to the hydroprocessing reactor. The distillation may be performed in either a batch or continuous mode. A single distillation operation can substantially decrease the plugging problem, but in one embodiment of the present invention, a double distillation process is performed. A double distillation process can remove substantially all of the contamination, and eliminate the plugging of the hydrotreating reactor such that hydroprocessing catalyst cycle times are restored to their desired two to four years or more duration.

Distillation of the hydrocarbon stream from a Fischer-Tropsch reactor to separate the stream into commercially viable products is known in the art. International Publication WO 00/11113 describes a vacuum distillation column with a plurality of distillation stages to separate usable wax products from a Fischer-Tropsch process effluent, with a 10 percent bottom stream used as a sump quench. EP 0579330 B1 and U.S. Pat. No. 5,486,542 describe a Fischer-Tropsch separation process using a wiped film evaporator to separate products. U.S. Pat. No. 2,852,546 describes an atmospheric distillation followed by a vacuum distillation of a Fischer-Tropsch wax to separate paraffins with melting points between 40 and 80° C. To the inventors' knowledge the prior art does not, however, describe the removal or control of contamination from the product stream from a Fischer-Tropsch reactor, nor does the prior art teach the recovery of a bottoms fraction from the distillation step, wherein the amount of the bottoms fraction is less than about 35 percent by volume of the filtered stream.

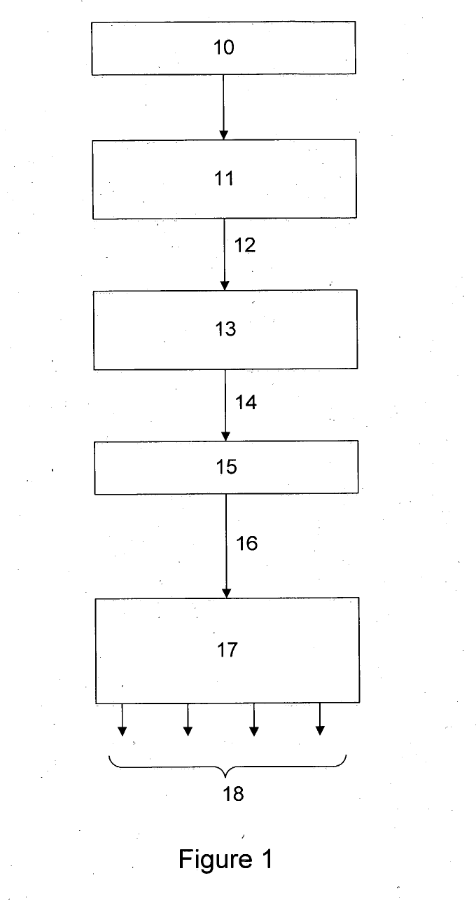

An overview of the embodiments of the present invention are shown schematically in FIG. 1. Referring to FIG. 1, a syngas 10 is passed to a Fischer-Tropsch reactor 11, which may contain an unfiltered slurry. Particulates in Fischer-Tropsch derived product 12 are removed in a conventional filtering process 13 to produce filtered stream 14. The conventional filtering process 13 may-take place inside the Fischer-Tropsch reactor 11, it may be integral to the Fischer-Tropsch reactor 11, or it may be external to Fischer-Tropsch reactor 11. After passing through the conventional filtering process 13, the Fischer-Tropsch derived hydrocarbon stream becomes a filtered stream 14 that is passed on to a distillation process 15 to form a distillate product stream 16. The distillate product stream 16 is then sent on to a hydroprocessing reactor 17, and an upgraded product 18 is recovered.

The following disclosure will first focus on the Fischer-Tropsch process itself, and then proceed to a discussion of hydroprocessing reactors and hydroprocessing conditions. Then the nature of contamination in general, and the specific problems associated with hydroprocessing catalyst bed plugging will be addressed, before turning to the present distillation embodiments.

The Fischer-Tropsch Synthesis

The Fischer-Tropsch process was adapted as a means to convert natural gas into liquid fuels. For this reason the process is also known as a “gas-to-liquids” process. The Fischer-Tropsch process reacts gaseous hydrocarbons (e.g. methane) with air (or oxygen, if the air is separated into its constituent components) over a first catalyst to create synthesis gas (also called “syngas”), which is substantially a mixture of carbon monoxide and hydrogen. The syngas is then converted into a mixture of liquid hydrocarbons using a second catalyst. The diesel boiling range material that is produced from this synthesis has many beneficial attributes, including a high cetane number, and essentially no sulfur, nitrogen, or aromatic content.

Catalysts and conditions for performing a Fischer-Tropsch synthesis are well known to those skilled in the art, and are described, for example, in EP 0 921 184 A1, the contents of which are hereby incorporated by reference in their entirety. In the Fischer-Tropsch synthesis process, liquid and gaseous hydrocarbons are formed by contacting a synthesis gas (syngas) 10 comprising a mixture of H₂ and CO with a Fischer-Tropsch catalyst under suitable reactive conditions. The Fischer-Tropsch synthesis is typically conducted in the reactor 11 at a temperature ranging from about 300 to 700° F. (149 to 371° C.), where a preferable temperature range is from about 400 to 550° F. (204 to 228° C.); a pressure ranging from about 10 to 600 psia (0.7 to 41 bars), preferably 30 to 300 psia (2 to 21 bars); and a catalyst space velocity ranging from about 100 to 10,000 cc/g/hr., where a preferable space velocity ranges from about 300 to 3,000 cc/g/hr.

The Fischer-Tropsch derived hydrocarbon stream (either unfiltered, 12, or conventionally filtered, 14) may range from C₁ to C₂₀₀₊, with a majority of the products in the C₅-C₁₀₀₊ range. In some embodiments of the present invention, the Fischer-Tropsch derived hydrocarbon stream is a C₃₊ product. In other embodiments, the Fischer-Tropsch derived hydrocarbon stream is a 550° F. product. A Fischer-Tropsch synthesis reaction may be conducted in a variety of reactor types including, for example, fixed bed reactors containing one or more catalyst beds, slurry reactors, fluidized bed reactors, or a combination of different reactor types. Such reaction processes and reactors are well known and documented in the literature.

In one embodiment of the present invention, the Fischer-Tropsch reactor 11 comprises a slurry type reactor. This type of reactor (and process) exhibit enhanced heat and mass transfer properties, and thus is capable of taking advantage of the strongly exothermic characteristics of a Fischer-Tropsch reaction. A slurry reactor produces relatively high molecular weight, paraffinic hydrocarbons when a cobalt catalyst is employed. Operationally, a syngas comprising a mixture of hydrogen (H₂) and carbon monoxide (CO) is bubbled up as a third phase through the slurry in the reactor, and the catalyst (in particulate form) is dispersed and suspended in the liquid. The mole ratio of the hydrogen reactant to the carbon monoxide reactant may range from about 0.5 to 4, but more typically this ratio is within the range of from about 0.7 to 2.75. The slurry liquid comprises not only the reactants for the synthesis, but also the hydrocarbon products of the reaction, since at least some of these products are in a liquid state at reaction conditions.

Suitable Fischer-Tropsch catalysts comprise one or more Group VIII catalytic metals such as Fe, Ni, Co, Ru and Re. The catalyst may include a promoter. In some embodiments of the present invention, the Fischer-Tropsch catalyst comprises effective amounts of cobalt and one or more of the elements Re, Ru, Fe, Ni, Th, Zr, Hf, U, Mg, and La on a suitable inorganic support material. In general, the amount of cobalt present in the catalyst is between about 1 and 50 weight percent, based on the total weight of the catalyst composition. Exemplary support materials include refractory metal oxides, such as alumina, silica, magnesia and titania, or mixtures thereof. In one embodiment of the present invention, the support material for a cobalt containing catalyst comprises titania. The catalyst promoter may be a basic oxide such as ThO₂, La₂O₃, MgO, and TiO₂, although promoters may also comprise ZrO₂, noble metals such as Pt, Pd, Ru, Rh, Os, and Ir; coinage metals such as Cu, Ag, and Au; and other transition metals such as Fe, Mn, Ni, and Re.

Useful catalysts and their preparation are known and illustrative, and nonlimiting examples may be found, for example, in U.S. Pat. No. 4,568,663.

Any C₅₊ hydrocarbon stream derived from a Fischer-Tropsch process may be suitably treated using the present process. Typical hydrocarbon streams include a C₅-700° F. stream and a waxy stream boiling above about 550° F., depending on the Fischer-Tropsch reactor configuration. In one embodiment of the present invention, a Fischer-Tropsch derived hydrocarbon stream 12, 14 is recovered directly from the reactor 11 without fractionation.

Hydroprocessing of the Distilled Fischer-Tropsch Reaction Products

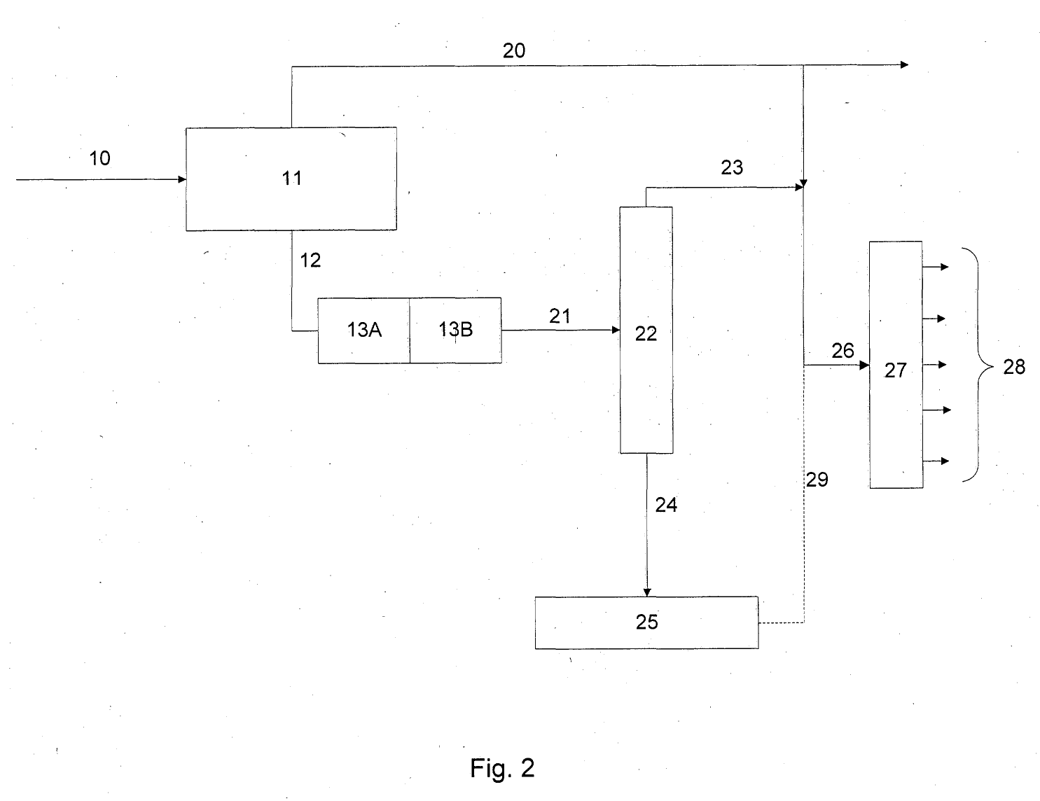

The product stream from the Fischer-Tropsch reactor may be subjected to a hydroprocessing step. This step may be carried out in the hydroprocessing reactor shown schematically at reference numerals 17 and 27 in FIGS. 1 and 2, respectively. The term “hydroprocessing” as used herein refers to any of a number of processes in which the products of the Fischer-Tropsch synthesis reaction produced by reactor 11 are treated with a hydrogen-containing gas; such processes include hydrodewaxing, hydrocracking, hydroisomerization, and hydrotreating.

As used herein, the terms “hydroprocessing,” “hydrotreating,” and “hydroisomerization” are given their conventional meaning, and describe processes that are known to those skilled in the art. Hydrotreating refers to a catalytic process, usually carried out in the presence of free hydrogen, in which the primary purpose is olefin saturation and oxygenate removal from the feed to the hydroprocessing reactor. Oxygenates include alcohols, acids, and esters. Additionally, any sulfur which may have been introduced when the hydrocarbon stream was contacted with a sulfided catalyst is also removed.

In general, hydroprocessing reactions may decrease the chain length of the individual hydrocarbon molecules in the feed being hydroprocessed (called “cracking”), and/or increase the isoparaffin content relative to the initial feed value. Referring to FIG. 2, the hydroprocessing conditions used in the hydroprocessing step 27 produce a product stream 28 that is rich in C₅-C₂₀ hydrocarbons, with reaction conditions selected to give the desired cold temperature properties (e.g., pour point, cloud point, and cold filter plugging point). Hydroprocessing conditions in step 27 which tend to form relatively large amounts of C₁₋₄ products are generally not preferred. Conditions which form C₂₀₊ products with a sufficient isoparaffin content to lower the melting point of the wax and/or heavy fraction (such that the particulates larger than 10 microns are more easily removed via conventional filtration) are also preferred.

In some embodiments of the present invention, it may be desirable to keep the amount of cracking of the larger hydrocarbon molecules to a minimum, and in these embodiments a goal of the hydroprocessing step 27 is the conversion of unsaturated hydrocarbons to either fully or partially hydrogenated forms. A further goal of the hydroprocessing step 27 in these embodiments is to increase the isoparaffin content of the stream relative to the starting value in the feed.

The hydroprocessed product stream 28 may optionally be combined with hydrocarbons from other sources such as gas oils, lubricant oil basestocks, high pour point polyalphaolefins, foots oil (oil that has been separated from an oil and wax mixture), synthetic waxes such as normal alpha-olefin waxes, slack waxes, de-oiled waxes, and microcrystalline waxes.

Hydroprocessing catalysts are well known in the art. See, for example, U.S. Pat. Nos. 4,347,121, 4,810,357, and 6,359,018 for general descriptions of hydroprocessing, hydroisomerization, hydrocracking, hydrotreating, etc., and typical catalysts used in such processes.

As discussed in U.S. Pat. No. 6,179,995, hydrotreating conditions may include a reaction temperature between 400 to 900° F. (204 to 482° C.), preferably 650 to 850° F. (343 to 454° C.); a pressure between 500 to 5000 psig (pounds per square inch gauge) (3.5 to 34.6 MPa), preferably 1000 to 3000 psig (7.0-20.8 MPa); a feed rate (LHSV) of 0.5 hr⁻¹ to 20 hr⁻¹ (v/v); and overall hydrogen consumption 300 to 2000 scf per barrel of liquid hydrocarbon feed (53.4-356 m³ H₂/m³ feed). The hydrotreating catalyst for the beds will typically be a composite of a Group VI metal or compound thereof, and a Group VIII metal or compound thereof supported on a porous refractory base such as alumina. Examples of hydrotreating catalysts are alumina supported cobalt-molybdenum, nickel sulfide, nickel-tungsten, cobalt-tungsten and nickel-molybdenum. Typically, such hydrotreating catalysts are presulfided. Non-sulfided and noble metal hydroprocessing catalysts (Ni, Pt, Pd, etc.) are also contemplated.

A hydrocracking reaction zone may be maintained at conditions sufficient to effect a boiling range conversion of a VGO (vacuum gas oil) feed to the hydrocracking reaction zone, so that the liquid hydrocrackate recovered from the hydrocracking reaction zone has a normal boiling point range below the boiling point range of the feed. Typical hydrocracking conditions include: reaction temperature, 400 to 950° F. (204 to 510° C.), preferably 650 to 850° F. (343 to 454° C.); reaction pressure 500 to 5000 psig (3.5 to 34.5 MPa), preferably 1500 to 3500 psig (10.4 to 24.2 MPa); LHSV, 0.1 to 15 hr⁻¹ (v/v), preferably 0.25-4 hr⁻¹; and hydrogen consumption 500 to 2500 scf (standard cubic feet) per barrel of liquid hydrocarbon feed (89.1 to 445 m³ H₂/m³ feed). The hydrocracking catalyst generally comprises a cracking component, a hydrogenation component and a binder. Such catalysts are well known in the art. The cracking component may include an amorphous silica/alumina phase and/or a zeolite, such as a Y-type or USY zeolite. The binder is generally silica or alumina. The hydrogenation component will be a Group VI, Group VII, or Group VIII metal or oxides or sulfides thereof, preferably one or more of molybdenum, tungsten, cobalt, or nickel, or the sulfides or oxides thereof. If present in the catalyst, these hydrogenation components generally make up from about 5 to about 40 percent by weight of the catalyst. Alternatively, platinum group metals, especially platinum and/or palladium, may be present as the hydrogenation component, either alone or in combination with the base metal hydrogenation components molybdenum, tungsten, cobalt, or nickel. If present, the platinum group metals will generally comprise from about 0.1 to 2 percent by weight of the catalyst.

Typical hydroisomerization conditions are well known in the literature and can vary widely. Isomerization processes are typically carried out at a temperature between about 200 and 700° F., preferably 300 to 650° F., with a LHSV between about 0.1 and 10 hr⁻¹, preferably between about 0.25 and 5 hr⁻¹. Hydrogen is employed such that the mole ratio of hydrogen to hydrocarbon is between about 1:1 and 15:1. Catalysts useful for isomerization processes are generally bifunctional catalysts that include a dehydrogenation/hydrogenation component and an acidic component. The acidic component may include one or more of the amorphous oxides such as alumina, silica or silica-alumina; a zeolitic material such as zeolite Y, ultra-stable Y, SSZ-32, Beta zeolite, mordenite, ZSM-5 and the like, or a non-zeolitic molecular sieve such as SAPO-11, SAPO-3 and SAPO-41. The acidic component may further include a halogen component, such as fluorine. The hydrogenation component hay be selected from the Group VIII noble metals such as platinum and/or palladium, from the Group VIII non-noble metals such as nickel and tungsten, and from the Group VI metals such as cobalt and molybdenum. If present, the platinum group metals will generally make up from about 0.1 to 2 percent by weight of the catalyst. If present in the catalyst, the non-noble metal hydrogenation components generally make up from about 5 to about 40 percent by weight of the catalyst.

The catalyst particles may have any shape known to be useful for catalytic materials, including spheres, fluted cylinders, prills, granules and the like. For non-spherical shapes, the effective diameter can be taken as the diameter of a representative cross section of the catalyst particles. The effective diameter of the zeolite catalyst particles ranges from about 1/32 inch to ¼ inch, and in some embodiments is preferably about 1/20 inch to ⅛ inch. Furthermore, the catalyst particles will further have a surface area ranging from about 50 to 500 m²/g.

Contamination, Filtering, and Hydroprocessing Catalyst Bed Plugging

Referring again to FIG. 2, the filtered stream 21 from the filtering steps 13A, 13B may cause plugging of catalyst beds in a hydroprocessing reactor due to particulates, particulate contamination, soluble contamination, fouling agents, and/or plugging precursors present in the filtered stream 21. The terms particulates, particulate contamination, soluble contamination, fouling agents, and plugging precursors will be used interchangeably in the present disclosure, but the phenomenon will in general be collectively referred to as “contamination,” keeping in mind that the entity that eventually plugs the hydroprocessing catalyst bed may be soluble in the feed at some time prior to the plugging event. The plugging event is a result of the contamination (which eventually takes a particulate form) being filtered out of the hydroprocessing feed by the catalyst beds of the hydroprocessing reactor 27. A typical Fischer-Tropsch process normally includes at least one filtering step 27 for removing catalyst particles from the waxy product produced by the Fischer-Tropsch reaction. These filters are designed to effectively remove particles having diameters greater than about 1 micron.

In an exemplary embodiment of the present invention, such a filtering step removes a substantial majority of the particulates having a size greater than or equal to about 1 micron in diameter, and a distillation step 22 is used to remove contamination, including particulates, soluble contamination, fouling agents, and plugging precursors from the filtered stream 21 such that plugging of the catalyst beds of the hydroprocessing reactor 27 is substantially avoided.

It may be beneficial to address contamination in general before discussing the details of the present distillation process. Contamination present in the filtered stream 21 can originate from a variety of sources, and, in general, methods are known in the art for dealing with at least some of its forms. These methods include, for example, separation, isolation, filtration, acid extraction and centrifugation.

In general, however, the presence of impurities such as mercaptans and other sulfur-containing compounds, halogen, selenium, phosphorus and arsenic contaminants, carbon dioxide, water, and/or non-hydrocarbon acid gases in the syngas 10 is undesirable, and for this reason they are preferably removed from the syngas feed before performing a synthesis reaction in the Fischer-Tropsch reactor 11. One method known in the art includes isolating the methane (and/or ethane and heavier hydrocarbon) component in the natural gas in a de-methanizer, and then de-sulfurizing the methane before sending it on to a conventional syngas generator to provide the synthesis gas 10. In an alternative prior art method ZnO guard beds may be used (and may even be preferred) for removing sulfur impurities.

Particulate contamination is usually addressed by filtering. Particulates such as catalyst fines that are produced in Fischer-Tropsch slurry or fluidized bed reactor 11 may be filtered out with commercially available filtering systems, such as a filtering step 13. If it is desired to remove particles larger than about 10 microns, a single filtering step is generally sufficient. For removing particles larger than 1 micron, multiple filtering steps maybe suitably employed.

The contaminants that are extracted from the Fischer-Tropsch derived hydrocarbon stream 12, according to embodiments of the present invention, may have both an organic component as well as an inorganic component. The organic component may have an elemental content that includes at least one of the elements carbon, hydrogen, nitrogen, oxygen, and sulfur (C, H, N, O, and S, respectively). The inorganic component may include at least one of the elements aluminum, cobalt, titanium iron, molybdenum, sodium, zinc, tin, and silicon (Al, Co, Ti, Fe, MO, Na, Zn, Sn, and Si, respectively).

Distillation of the Fischer-Tropsch Hydrocarbon Products

The use of a distillation step to fractionate the components of a Fischer-Tropsch product stream is known in the art. Such a fractionation step has been used to prepare commercially viable hydrocarbon products for market. To the inventors' knowledge, however, distillation has not been used previously to remove contamination from a Fischer-Tropsch derived product stream, and distillation of a Fischer-Tropsch derived product stream to substantially avoid plugging of the hydroprocessing catalyst beds used in a subsequent process is not known in the art.

Publication WO/00/11113 describes a Fischer-Tropsch vacuum distillation column having a plurality of distillation stages for separating out usable wax products from the remainder of the product stream. The distillation produces a 10 percent by volume bottoms stream that may be used as a sump quench. U.S. Pat. No. 5,486,542 and EP 0579330 B1 describe a Fischer-Tropsch separation process that uses a wiped film evaporator to separate out Fischer-Tropsch products. U.S. Pat. No. 2,852,546 describes an atmospheric distillation followed by a vacuum distillation of a Fischer-Tropsch wax to separate out paraffins having melting points between about 40 and 80° C.

The prior art does not teach or disclose the need for methods of contamination removal from a Fischer-Tropsch derived hydrocarbon stream by distillation such that the contamination is concentrated into a particular fraction for isolation from the hydrocarbon stream. Nor does the prior art teach a distillation process whereby a fraction containing the isolated contamination has a volume less than about 35 percent of the total product stream exiting the Fischer-Tropsch reactor.

Preferably, the amount of commercially viable product recovered from the Fischer-Tropsch process is 85 percent or greater, and the fraction containing the isolated contamination is less than about 15 percent by volume of the product stream exiting the Fischer-Tropsch reactor. More preferably, the amount of commercially viable product recovered is 90 percent or greater, and the fraction containing the isolated contamination is less than about 10 percent by volume. Still more preferably, the amount of commercially viable products recovered is 95 percent or greater, and the fraction containing the isolated contamination is less than about 5 percent by volume.

The inventors have discovered that distillation of the Fischer-Tropsch derived product stream prior to hydroprocessing substantially eliminates the contamination that causes plugging of the catalyst beds of the hydroprocessing reactor. The distillation may be either a batch or continuous process. Furthermore, the distillation procedure may comprise a one-step, two-step, or more than two-step distillation process.

The configuration of the distillation will depend on the manner in which the Fischer-Tropsch process was carried out (which will also influence the yield), and the molecular weight distribution of the recovered products. In general, the Fischer-Tropsch product will be a full boiling range product, having an initial boiling point corresponding to that of propane (i.e. C₃₊), or it will be a substantially waxy product, having an initial boiling point ranging from about 550 to 750° F.

A more detailed look at embodiments of the present invention, particularly with regard to distillation, is given in FIG. 2. Referring to FIG. 2, a syngas 10 is passed to a Fischer-Tropsch synthesis reactor 11. The effluent from the Fischer-Tropsch reactor includes a condensate 20 and an unfiltered or coarsely filtered (wherein coarsely filtered means particles 10 microns or greater in size are substantially removed) product stream 12, the stream 12 comprising heavier molecular weight hydrocarbons than the condensate 20. The condensate 20 is a lighter molecular weight fraction that may initially comprise a gas which condenses to a liquid once that fraction has exited the reaction zone. Thus, the condensate 20 is unlikely to contain any of the plugging agents, particulates, contamination, plugging precursors, and/or undesired species. Therefore, it may not be necessary to filter the condensate stream 20.

In one embodiment of the present invention, the filtering process 13 depicted in FIG. 2 may comprise two or individual filtering steps. For example, a first filtering step removes a substantial portion of the particulate contamination having sizes greater than about 10 microns in diameter. This first filtering step may take place inside the Fischer-Tropsch reactor. A second filtering step removes at least a portion of the particulate contamination which is smaller than 10 micron in diameter, generally down to 1 micron in diameter, and in some cases down to particulates as small as 0.1 microns in diameter or smaller.

In addition to filtering the Fischer-Tropsch derived hydrocarbon stream 12, the filtered stream 21 may also be treated with an acid (this step is not shown in FIG. 2) to extract contamination. The acid extraction step may be performed either immediately before or immediately after the filtering step 13.

The filtered stream 21 is then passed to a distillation step 22, which produces at least a light fraction 23 and a bottoms stream 24. Bottoms stream 24 contains a substantial portion of the contamination present in the filtered stream 21. This bottoms stream 24 may be treated 25 to remove the contamination. Suitable treatments include additional conventional filtering, contacting at least a portion of the bottoms stream with an aqueous acid or passing at least a portion of the bottoms stream through a column of solid adsorbent material for removing the contamination. Alternatively, the bottoms stream may be used as fuel, blended with crude oil, used in other processes which can tolerate contaminated hydrocarbons, or discarded. Treated bottoms stream may be sent as a stream 29 to the hydroprocessing reactor 27. In a specific embodiment, the stream 29, light fraction 23, and condensate 20 may be upgraded in the hydroprocessing reactor 27 either separately, as a combined stream 26, or in any number of other combinations (i.e., 20 and 23, or 23 and 29).

In a preferred distillation step of the present process, less than 35 percent by volume and more preferably less than 15, 10, and 5 percents by volume of the filtered stream 21 is recovered as bottoms fraction 25. It is expected that, to achieve such high concentration of the contamination in the bottoms fraction, at least one vacuum distillation step will be required in the distillation process 22, with the bottoms stream 24 having an initial boiling point of greater than about 1,000° F., Of course, it will be noted by one of ordinary skill in the art that the distillation process, conditions, and procedures could have been adjusted to achieve any desired quantity of the bottoms fractions relative to the feed, and in some embodiments the volume of the bottoms fraction selected to isolate the contamination may even be smaller than 5 percent by volume. This unusually high ratio of the filtered stream 21 to the bottoms fraction 25 results from the ability of the present embodiments to concentrate the contamination in a bottoms stream.

In embodiments of the present invention, the filtering steps remove particulate contamination that is derived from the Fischer-Tropsch catalyst used in reactor 11. In other embodiments, the distillation step 22 removes both particulate and soluble contamination. Both forms of contamination (soluble and particulate) may be derived from the Fischer-Tropsch catalyst used in reactor 11, and may have a metallic component comprising a material selected from the group consisting of aluminum, cobalt, titanium, and iron.

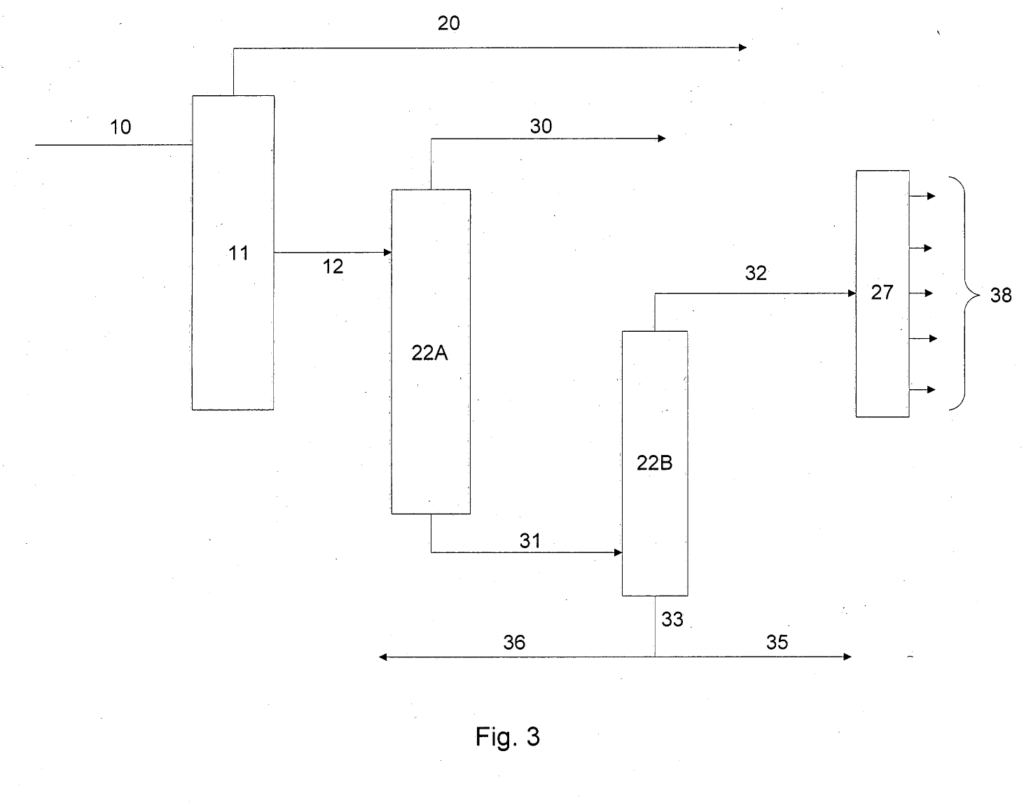

FIG. 3 illustrates the present process, with the distillation process 22 employing multiple distillations steps. In FIG. 3, a syngas 10 is passed to a Fischer-Tropsch reactor 11, and the effluent from the Fischer-Tropsch reactor 11 includes a condensate 20 and a product stream 12. Stream 12 is passed to a first distillation step 22A, which comprises a vacuum distillation, and preferably an atmospheric distillation followed by a vacuum distillation. The first distillation step 22A produces a first overhead stream 30 and a first bottoms stream 31. The initial boiling point of stream 31 is in the range of about 800 to 950° F. First overhead stream 30 will generally be treated using conventional processing, such as hydroprocessing, or it may be recovered for other uses, such as fuels, petrochemical feedstocks, and the like. In this specific example, it is shown as being combined with other streams in the process for hydroprocessing. The first bottoms stream 31 is passed on to a second distillation step, in this case a vacuum distillation step 22B.

The second distillation step 22B also produces a light and heavy stream, in this case a second overhead stream 32 and a second bottoms stream 33 having an initial boiling point of greater than about 1000° F. While the second overhead stream 32 may then be passed to the hydroprocessing reactor 27, the second bottoms stream 33 may either be dispensed in crude oil in a step 35, passed to a recycle operation 36 for further processing, used as a fuel or as some other material for which a tar is appropriate, or passed to yet another distillation column (not depicted in the exemplary two-step distillation process of FIG. 3). In some embodiments of the present invention, the second bottoms stream is less than about 15 percent by volume of the Fischer-Tropsch derived hydrocarbon stream 12.

The multiple distillation process disclosed above, which in an exemplary embodiment of a two-step distillation process is contemplated by the inventors to substantially eliminate plugging of the hydroprocessing reactor 27, thus extending the cycle time of the hydroprocessing catalyst to at least a two year duration. Additional advantages include a recovery of at least a 70 percent by volume of commercially viable product from the Fischer-Tropsch process.

### EXAMPLES

The following examples illustrate various ways in which a distillation process may be used to treat a Fischer-Tropsch derived product stream before sending that stream on to hydroprocessing. The following examples are given for the purpose of illustrating embodiments of the present invention, and should not be construed as being limitations on the scope or spirit of the instant invention.

### Example 1

### Double Batch Distillation of a Fischer-Tropsch Product Stream

This example gives the results of a double batch distillation process performed on a Fischer-Tropsch derived paraffinic product stream to remove contamination, wherein the second distillation process is a vacuum distillation. The levels of a variety of different elements (mostly metals) were measured in the Fischer-Tropsch wax prior to distillation, and then in two of the cuts from the first distillation, and the bottoms fraction from the second distillation.

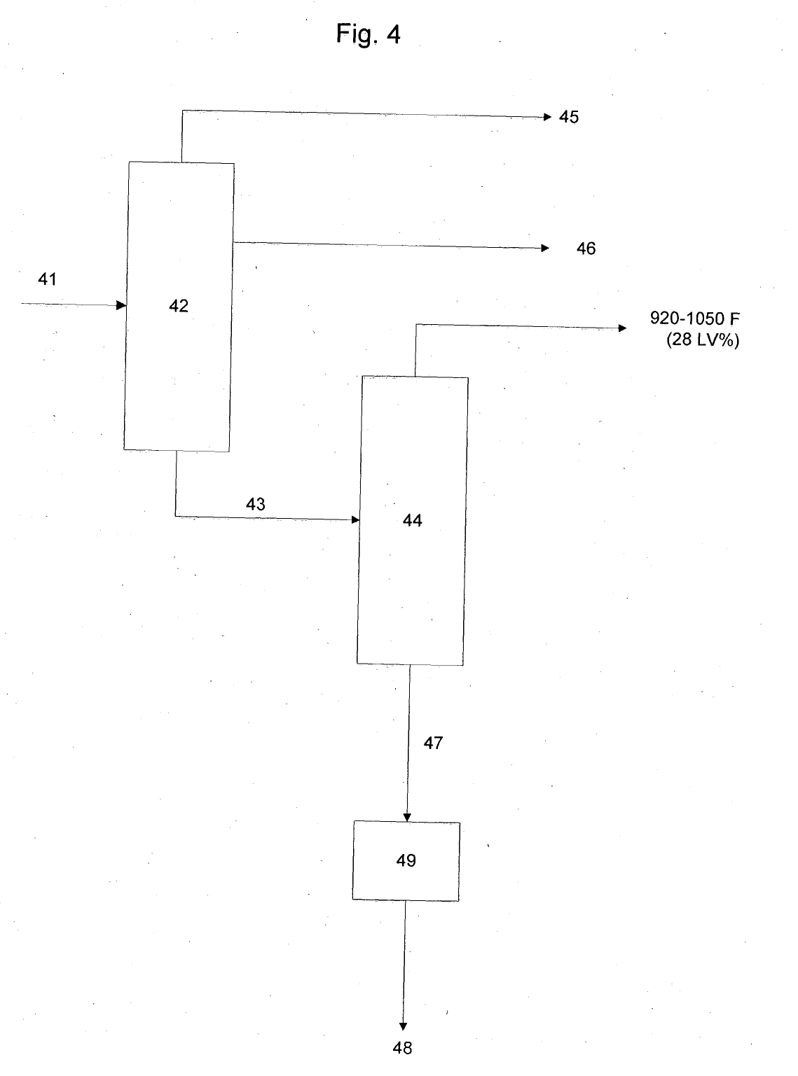

The identity of the samples is shown in FIG. 4, along with an overview of the double batch distillation process used in Example 1. Referring to FIG. 4, a Fischer-Tropsch wax feed 41 is charged to a first distillation step 42, and the bottoms fraction 43 from the first distillation step 42 charged to a second distillation step 44. The Fischer-Tropsch wax feed is sample 41. Sample 45 is a 750° F.− stream that originates from the first distillation step 42, and sample 46 is a 750-920° F. fraction also from the first distillation step 42. The third sample is a bottoms fraction 47 from the second distillation step 44. A fourth sample 48 comprises the bottoms fraction 47 after it had been passed through a 0.1 micron filter 49.

Table I shows the contamination levels in parts per million (ppm) as measured by inductively coupled plasma atomic emission spectroscopy (ICP-AES):

TABLE I

| | | 750° F.- | 750-920° F. | Bottoms | Bottoms |
|---|---|---|---|---|---|
| | | stream | fraction from | fraction from | fraction 47 |
| Contaminant | FT | from | the first | the second | filtered |
| (in ppm, as | wax | the first | distillation | distillation | through a |
| measured by | feed | distalla- | step | step | 0.1 micron |
| ICP-AES) | (41) | tion (45) | (46) | (47) | filter (48) |
| Al | 29 | <1.3 | <1.3 | 248 | 221 |
| Ca | <0.5 | <0.5 | <0.5 | <0.4 | 0.9 |
| Co | 2.4 | <0.1 | <0.1 | 22.7 | 19.9 |
| Fe | 0.5 | 0.6 | 0.7 | 9.2 | 8.2 |
| K | <2.3 | <2.4 | <2.3 | <2.2 | <1.7 |
| Mg | <0.5 | <0.5 | <0.5 | <0.5 | 0.3 |
| Mo | <1.1 | <1.2 | <1.2 | <1.1 | <1.1 |
| Na | 0.6 | 0.4 | 0.6 | 1.6 | 1.9 |
| Ni | 0.1 | 0.1 | <0.1 | 0.5 | 0.4 |
| Si | 1 | <0.3 | 0.4 | 8.1 | 5.5 |
| Sn | 0.3 | 1.8 | 2.8 | 10.1 | 8.5 |
| Ti | <0.2 | <0.2 | <0.2 | <0.2 | <0.1 |
| V | <0.7 | <0.7 | <0.7 | <0.7 | <0.7 |
| Zn | 0.1 | 0.2 | 0.1 | 0.9 | <1.3 |

Table I clearly shows the ability of the distillation process to concentrate and isolate the contamination into a separate fraction, which in this exemplary process is the bottoms fraction of a second (vacuum) distillation step. It will also be noted in FIG. 4 that the amount of the bottoms fraction 47 relative to the amount of the filtered Fischer-Tropsch wax feed 41 was 12 liquid volume percent (LV %) in this exemplary procedure; in other words, of the total amount of Fischer-Tropsch wax being fed to the distillation process, about 12 percent by volume ended up in the bottoms fraction selected to remove the contamination.

It may also be seen from Table I that the contamination passes through a 0.1 micron filter, as the levels of the contaminants in sample 48 (filtered) are essentially as great as they are in sample 47 (unfiltered).

### Example 2

### Continuous Distillation of a Fischer-Tropsch Product Stream

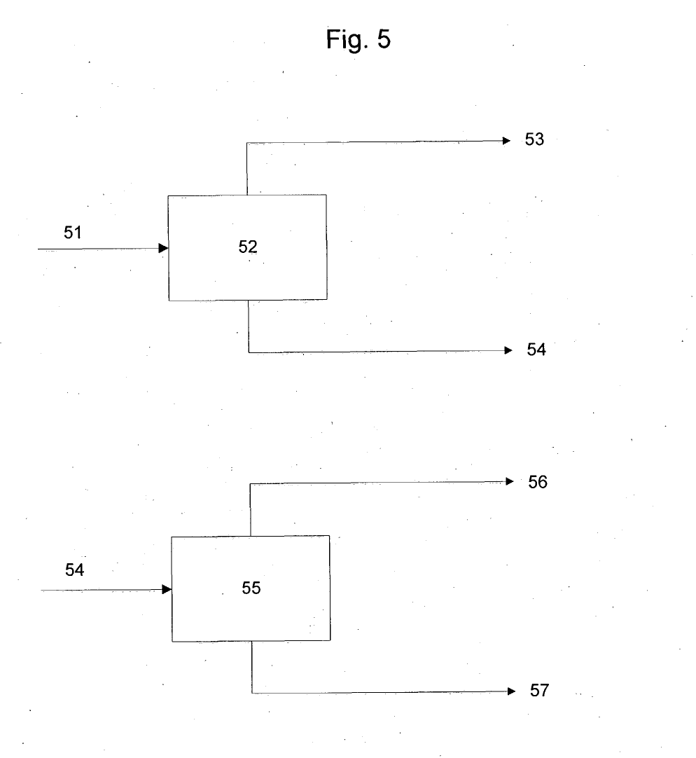

This example gives the results of a continuous distillation process performed on a Fischer-Tropsch derived paraffinic product stream to remove contamination, wherein the continuous distillation is performed as two separate distillation steps. The samples are identified in FIG. 5. Referring to FIG. 5,.a Fischer-Tropsch wax feed 51 is charged to a first distillation step 52 which produces a St-840° F. stream 53 and an 840° F.+ stream 54. The 840° F.+ stream 54 is continuously charged to a second distillation step 55, which in turn produces an 840-960° F. stream 56 and a 960° F.+ stream 57.

Table II shows the contamination levels in parts per million (ppm), again as measured by inductively coupled plasma atomic emission spectroscopy (ICP-AES), in this example of a continuous distillation:

TABLE II

| Contaminant | | | |
|---|---|---|---|
| (in ppm, as | | | |
| measured by | St-840° F. | 840-960° F. | 960° F.+ |
| ICP-AES) | (53) | (56) | (57) |
| Al | <1.1 | <1.1 | 146 |
| Ca | <0.5 | <0.4 | 0.8 |
| Co | <0.4 | <0.1 | 10.9 |
| Fe | 1 | 0.8 | 7.5 |
| K | <1.6 | <1.6 | <1.4 |
| Mg | 0.1 | <0.1 | 0.2 |
| Mo | <1.1 | <1.1 | <1.0 |
| Na | <1.1 | <1.1 | 2 |
| Ni | <0.1 | <0.1 | 0.2 |
| Si | <0.9 | 1 | 5.2 |
| Sn | 2.2 | 1.7 | <1.0 |
| Ti | 0.1 | <0.1 | 3.5 |
| V | <0.7 | <0.6 | <0.6 |
| Zn | 1.5 | 1.5 | 1.1 |

Table II also shows the ability of the distillation process to concentrate and isolate the contamination into a separate stream, which in this exemplary process is the bottoms stream of the second distillation step of this continuous distillation process, the 960° F.+ stream 57. The amounts of the streams 53, 56, and 57, as a percentage of the filtered Fischer-Tropsch wax feed, were 50.04, 19.79, and 30.17 weight percent, respectively. Thus, in this exemplary continuous distillation, the distillation conditions were designed such that the amount of the bottoms fraction designed to concentrate and isolate the contamination was about 35 LV % of the filtered Fischer-Tropsch wax feed 51.

All of the publications, patents and patent applications cited in this application are herein incorporated by reference in their entirety to the same extent as if the disclosure of each individual publication, patent application or patent was specifically and individually indicated to be incorporated by reference in its entirety.

Many modifications of the exemplary embodiments of the invention disclosed above will readily occur to those skilled in the art. Accordingly, the invention is to be construed as including all structure and methods that fall within the scope of the appended claims.

1. A method of removing contamination from a Fischer-Tropsch derived hydrocarbon stream, the method comprising:
a) filtering a Fischer-Tropsch derived hydrocarbon stream to produce a filtered hydrocarbon stream;
b) passing the filtered hydrocarbon stream to at least one distillation step to remove contamination from the filtered hydrocarbon stream, the distillation step producing a distillate product stream and a bottoms fraction, wherein the contamination is substantially concentrated in the bottoms fraction; and
c) recovering the bottoms fraction from the distillation step, wherein the amount of the bottoms fraction is less than about 35 percent by volume of the filtered hydrocarbon stream.

2. The method of claim 1, wherein the amount of the bottoms fraction is less than about 15 percent by volume of the filtered hydrocarbon stream.

3. The method of claim 1, wherein the filtering step removes particulates having an average size greater than or equal to about 10 microns.

4. The method of claim 1, wherein the filtering step removes contamination having an average size greater than or equal to about 1 micron.

5. The method of claim 1, wherein the distillation step is carried out in a vacuum distillation column.

6. The method of claim 1, wherein the contamination is derived from a Fischer-Tropsch catalyst.

7. The method of claim 1, wherein the distillation step removes soluble contamination from the Fischer-Tropsch derived hydrocarbon stream, the soluble contamination being derived from a Fischer-Tropsch catalyst.

8. The method of claim 7, wherein the soluble contamination comprises a material selected from the group consisting of aluminum, cobalt, titanium and iron.

9. The method of claim 1, wherein at least about 70 percent by weight of the contamination present in the filtered hydrocarbon stream is isolated in the bottoms fraction.

10. The method of claim 1, wherein at least about 85 percent by weight of the contamination present in the filtered hydrocarbon stream is isolated in the bottoms fraction.

11. The method of claim 1, wherein the Fischer-Tropsch derived hydrocarbon stream is a C₃₊ product.

12. The method of claim 1, further including passing the distillate product stream to a hydroprocessing reactor having a hydroprocessing catalyst.

13. The method of claim 1, wherein the distillation step comprises a first distillation step and a second distillation step, the first distillation step producing a first overhead stream and a first bottoms stream, and wherein the second distillation step produces a second overhead stream and a second bottoms stream.

14. The method of claim 13, wherein the first overhead stream has a range of boiling points less than about 800 to 950° F., and the first bottoms stream has a range of boiling points greater than about 800 to 950° F.

15. The method of claim 13, wherein the first overhead stream is passed to the hydroprocessing reactor, and the first bottoms stream is passed to the second distillation step.

16. The method of claim 13, wherein the second distillation step comprises a vacuum distillation, and the second bottoms stream has an initial boiling point of greater than about 1000° F.

17. The method of claim 13, wherein the second overhead stream is passed to the hydroprocessing reactor.

18. The method of claim 13, wherein the second bottoms stream is less than about 15 percent by volume of the Fischer-Tropsch derived hydrocarbon stream.

19. The method of claim 13, further including the step of treating the second bottoms stream with a treatment selected from the group consisting of dispensing the second bottoms stream in crude oil, passing the second bottoms stream to a third distillation step, processing the second bottoms stream into a fuel, and recycling the second bottoms stream in a recycling operation.
